SEQUENCE LISTING (30 sequences)

SEQ ID NO: 1 - protein, 27 aa, Artificial
  Synthetic
IPLRGAFINGRWDSQCHRFSNGAIACA

SEQ ID NO: 2 - protein, 37 aa, Artificial
  Synthetic
SIFSLFKMGAKALGKTLLKQAGKAGAEYAACKATNQC

SEQ ID NO: 3 - protein, 34 aa, Artificial
  Synthetic
SFVTKLKDVAIGVAKGAGLGILKTLTCKLDNSCA

SEQ ID NO: 4 - protein, 34 aa, Artificial
  Synthetic
SFITKLKDVAIGVAKGAGLGILKTLTCKLDNSCA

SEQ ID NO: 5 - protein, 27 aa, Artificial
  Synthetic
APLRGAFINGRWDSQCHRFSNGAIACA

SEQ ID NO: 6 - protein, 27 aa, Artificial
  Synthetic
IALRGAFINGRWDSQCHRFSNGAIACA

SEQ ID NO: 7 - protein, 27 aa, Artificial
  Synthetic
IPARGAFINGRWDSQCHRFSNGAIACA

SEQ ID NO: 8 - protein, 27 aa, Artificial
  Synthetic
IPLAGAFINGRWDSQCHRFSNGAIACA

SEQ ID NO: 9 - protein, 27 aa, Artificial
  Synthetic
IPLRAAFINGRWDSQCHRFSNGAIACA

SEQ ID NO: 10 - protein, 27 aa, Artificial
  Synthetic
IPLRGAAINGRWDSQCHRFSNGAIACA

SEQ ID NO: 11 - protein, 27 aa, Artificial
  Synthetic
IPLRGAFANGRWDSQCHRFSNGAIACA

SEQ ID NO: 12 - protein, 27 aa, Artificial
  Synthetic
IPLRGAFIAGRWDSQCHRFSNGAIACA

SEQ ID NO: 13 - protein, 27 aa, Artificial
  Synthetic
IPLRGAFINARWDSQCHRFSNGAIACA

SEQ ID NO: 14 - protein, 27 aa, Artificial
  Synthetic
IPLRGAFINGAWDSQCHRFSNGAIACA

SEQ ID NO: 15 - protein, 27 aa, Artificial
  Synthetic
IPLRGAFINGRADSQCHRFSNGAIACA

SEQ ID NO: 16 - protein, 27 aa, Artificial
  Synthetic
IPLRGAFINGRWASQCHRFSNGAIACA

SEQ ID NO: 17 - protein, 27 aa, Artificial
  Synthetic
IPLRGAFINGRWDAQCHRFSNGAIACA

SEQ ID NO: 18 - protein, 27 aa, Artificial
  Synthetic
IPLRGAFINGRWDSACHRFSNGAIACA

SEQ ID NO: 19 - protein, 27 aa, Artificial
  Synthetic
IPLRGAFINGRWDSQAHRFSNGAIACA

SEQ ID NO: 20 - protein, 27 aa, Artificial
  Synthetic
IPLRGAFINGRWDSQCARFSNGAIACA

SEQ ID NO: 21 - protein, 27 aa, Artificial
  Synthetic
IPLRGAFINGRWDSQCHAFSNGAIACA

SEQ ID NO: 22 - protein, 27 aa, Artificial
  Synthetic
IPLRGAFINGRWDSQCHRASNGAIACA

SEQ ID NO: 23 - protein, 27 aa, Artificial
  Synthetic
IPLRGAFINGRWDSQCHRFANGAIACA

SEQ ID NO: 24 - protein, 27 aa, Artificial
  Synthetic
IPLRGAFINGRWDSQCHRFSAGAIACA

SEQ ID NO: 25 - protein, 27 aa, Artificial
  Synthetic
IPLRGAFINGRWDSQCHRFSNAAIACA

SEQ ID NO: 26 - protein, 27 aa, Artificial
  Synthetic
IPLRGAFINGRWDSQCHRFSNGAAACA

SEQ ID NO: 27 - protein, 27 aa, Artificial
  Synthetic
IPLRGAFINGRWDSQCHRFSNGAIAAA

SEQ ID NO: 28 - DNA, 30 bp, Artificial
  Synthetic
aagcagtggtatcaacgcagagtacgcggg

SEQ ID NO: 29 - DNA, 57 bp, Artificial
  Synthetic
  n (56)..(56): n is A, G, or C
  misc_feature (56)..(57): n is a, c, g, t or u
  n (57)..(57): n is A, C, G, or T
aagcagtggtatcaacgcagagtacttttttttttttttttttttttttttttttnn

SEQ ID NO: 30 - DNA, 23 bp, Artificial
  Synthetic
aagcagtggtatcaacgcagagt

(19) United States
(12) Patent Application Publication
(10) Pub. No.: US 2019/0367567 A1
(43) Pub. Date: Dec. 5, 2019

(54) Peptides and Uses for Managing Viral Infections

(71) Applicant: Emory University, Atlanta, GA (US)

(72) Inventors: Joshy Jacob, Snelville, GA (US); David Holthausen, Atlanta, GA (US); Song Hee Lee, Atlanta, GA (US); Sanil George, Trivandrum (IN)

(21) Appl. No.: 16/466,258

(22) PCT Filed: Dec. 1, 2017
(86) PCT No.: PCT/US2017/064316

§ 371 Date: Jun. 3, 2019

Related U.S. Application Data
(60) Provisional application No. 62/429,231, filed on Dec. 2, 2016.
(60) Provisional application No. 62/466,006, filed on Mar. 2, 2017.

(51) Int. Cl. C07K 14/46; A61P 31/16

(52) U.S. Cl. CPC C07K 14/463; A61K 38/00; A61P 31/16

(57) ABSTRACT

It has been discovered that certain peptides are useful for managing certain viral infections. Thus, this disclosure relates to the use of peptides reported herein for the prevention or treatment of viral infections such as influenza infections. In certain embodiments, this disclosure relates to peptides, variants, or derivatives having sequences disclosed herein and pharmaceutical compositions comprising the same.

### CROSS-REFERENCE TO RELATED APPLICATIONS

This application claims the benefit of U.S. Provisional Application No. 62/429,231 filed Dec. 2, 2016 and U.S. Provisional Application No. 62/466,006 filed Mar. 2, 2017. The entirety of each of these applications is hereby incorporated by reference for all purposes.

### INCORPORATION-BY-REFERENCE OF MATERIAL SUBMITTED AS A TEXT FILE VIA THE OFFICE ELECTRONIC FILING SYSTEM (EFS-WEB)

The Sequence Listing associated with this application is provided in text format in lieu of a paper copy, and is hereby incorporated by reference into the specification. The name of the text file containing the Sequence Listing is 16134PCT ST25.txt. The text file is 10 KB, was created on Dec. 1, 2017, and is being submitted electronically via EFS-Web.

### BACKGROUND

Influenza is a common recurring human respiratory virus. Current vaccination strategies offer protection; however, pandemic outbreaks occur unexpectedly limiting the ability to develop vaccines in a timely manner. For seasonal influenza viruses, mismatches frequently occur between the vaccine and the circulating strains. In addition, the emergence of drug-resistant influenza viruses is a major concern. Thus, there is a pressing need to develop new therapies for the management of influenza infections.

Skalickova et al. report the use of anti-viral peptides against influenza virus. Viruses, 2015, 7(10): 5428-5442. See also Kumar et al. First report of Lividin and Spinulosain peptides from the skin secretion of an Indian frog. Acta Biol Hung, 2016, 67, 121-124.

References cited herein are not an admission of prior art.

### SUMMARY

It has been discovered that certain peptides are useful for managing certain viral infections. This disclosure relates to the use of peptides reported herein for the prevention or treatment of viral infections such as influenza infections. In certain embodiments, this disclosure relates to peptides, variants, or derivatives having sequences disclosed herein and pharmaceutical compositions comprising the same.

In certain embodiments, the peptides comprise or consist of SEQ ID NO: 1-27, or variants thereof. In certain embodiments, the variants have one, two, three, or more amino acid substitutions, insertions, or deletions. In certain embodiments, the peptides are produced synthetically or recombinantly. In certain embodiments, the peptides have at least one non-naturally occurring amino acid substitution, addition, or deletion. In certain embodiments, the amino acid substitutions are conserved substitutions.

In certain embodiments, the disclosure contemplates peptides disclosed herein having at least one molecular modification, e.g., such that the peptide contains a non-naturally amino acid. In certain embodiments, the disclosure contemplates a non-naturally occurring derivative of a peptide having SEQ ID NO: 1-27, variants, or derivatives thereof. In certain embodiments, the disclosure contemplates a derivative in the form of a prodrug. In certain embodiments, the disclosure contemplates a derivative wherein an amino, carboxyl, hydroxyl, or thiol group in a peptide disclosed herein is substituted. In certain embodiments, the disclosure contemplates peptides disclosed herein having a label, e.g., fluorescent or radioactive.

In certain embodiments, the disclosure contemplates a peptide having at least 50%, 60%, 70%, 80%, 90%, 95% sequence identity or similarity to SEQ ID NO: 1-27, and contains at least one substitution and/or modification relative to SEQ ID NO: 1-27 such that the entire peptide is not naturally occurring, e.g., one or more amino acids have been changed relative to the natural sequence.

In certain embodiments, the disclosure relates to recombinant vectors comprising a nucleic acid encoding peptide disclosed herein. In certain embodiments, the disclosure relates to expression systems comprising a recombinant vector comprising a nucleic acid encoding peptide disclosed herein. In certain embodiments, the disclosure relates to cells comprising a recombinant vector comprising a nucleic acid encoding peptide disclosed herein. In certain embodiments, the disclosure relates to a vector comprising the nucleic acid encoding a peptide disclosed herein and a heterologous nucleic acid sequence.

In certain embodiments, the disclosure relates to a nucleic acid encoding a polypeptide disclosed herein wherein the nucleotide sequence has been changed to contain at least one non-naturally occurring substitution and/or modification relative to the naturally occurring sequence, e.g., one or more nucleotides have been changed relative to the natural sequence. In certain embodiments, the disclosure relates to a nucleic acid encoding a polypeptide disclosed herein further comprising a label.

In certain embodiments, the disclosure relates to pharmaceutical compositions comprising a peptide having SEQ ID NO: 1-27, variants, or derivatives thereof and a pharmaceutically acceptable excipient. In certain embodiments, the pharmaceutical composition is in the form of a capsule, tablets, pill, powder, granule, or gel. In certain embodiments, the pharmaceutical composition is in the form of a sterilized pH buffered aqueous salt solution, or in the form of a container configured to spray a liquid, or in the form of a sealed container with a propellant. In certain embodiments, the disclosure contemplates the preparation of a medicament disclosed herein for useful for treating or preventing viral infections. In certain embodiments, the pharmaceutical compositions is in solid form surrounded by an enteric coating. In certain embodiments, the pharmaceutical compositions a pharmaceutically acceptable excipient is a solubilizing agent.

In certain embodiments, the disclosure relates to methods of treating or preventing a viral infection, such as an influenza infection, comprising administering an effective amount of a pharmaceutical composition comprising a peptide having SEQ ID NO: 1-27, variants, or derivatives thereof to a subject in need thereof. In certain embodiments, the subject is at risk of, exhibiting symptoms of, or diagnosed with a viral infection. In certain embodiments, the subject is at risk of, exhibiting symptoms of, or diagnosed with an influenza infection of influenza subtype H1.

In certain embodiments, the peptides having SEQ ID NO: 1-27, variants, or derivatives thereof are administered in combination with another or second therapeutic agent or antiviral agent. In certain embodiments, the second anti-viral agent is oseltamivir, zanamivir, and/or peramivir. In certain embodiments, the antiviral agent(s) is abacavir, acyclovir, acyclovir, adefovir, amantadine, amprenavir, ampligen, arbidol, atazanavir, atripla, boceprevir, cidofovir, combivir, complera, darunavir, delavirdine, didanosine, docosanol, dolutegravir, edoxudine, efavirenz, emtricitabine, enfuvirtide, entecavir, famciclovir, fomivirsen, fosamprenavir, foscarnet, fosfonet, ganciclovir, ibacitabine, imunovir, idoxuridine, imiquimod, indinavir, inosine, interferon type III, interferon type II, interferon type I, lamivudine, lopinavir, loviride, maraviroc, moroxydine, methisazone, nelfinavir, nevirapine, nexavir, oseltamivir, peginterferon alfa-2a, penciclovir, peramivir, pleconaril, podophyllotoxin, raltegravir, ribavirin, rimantadine, ritonavir, pyramidine, saquinavir, stavudine, stribild, tenofovir, tenofovir disoproxil, tenofovir alafenamide fumarate, tipranavir, trifluridine, trizivir, tromantadine, truvada, valaciclovir, valganciclovir, vicriviroc, vidarabine, viramidine, zalcitabine, zanamivir, zidovudine, and combinations thereof.

### BRIEF DESCRIPTION OF THE DRAWINGS

FIG. 1 illustrates the sequences of peptides (SEQ ID NO: 1-4).

FIG. 2A illustrates the sequences of Urumin variants (SEQ ID NO: 5-27).

FIG. 2B shows data indicating alanine mutants and Urumin peptide are not toxic to human red blood cells.

FIG. 3A shows data screening of 32 peptides isolated from the skin of H. bahuvistara against A/PuertoRico/8/1934 H1N1 influenza virus by plaque assay. Frog peptides were used at a concentration of 10 μM except peptides 11, 26, 28, 29, 32 that were tested at lower concentration due to hemolytic activity at 10 μM. Peptides highlighted reduced viral titers in comparison to OVA control peptide.

FIG. 3B shows toxicity of peptides that inhibited A/PR/8/34 virus by human RBC hemolysis. Peptide toxicity was compared to lysis induced by 0.1% Triton X-100 that was set as 100% cell lysis, and PBS as 0% lysis.

FIG. 3C shows data for Urumin IC50. Tested by graded concentrations of Urumin (0.6-320 μM) against PR8 virus by plaque assay.

FIG. 4A shows testing data of 100 μM Urumin or control OVA peptide against 8H1N1 influenza viruses from 1934-2013 and 4H3N2 influenza viruses from 1968-2003 by plaque assay.

FIG. 4B shows testing data of 100 μM Urumin and OVA control peptides against four reassortant PR8 viruses that shared all 6 internal gene segments and differed only in HA and NA segments (H1N1, H1N2, H3N1, H3N2).

FIG. 4C shows testing data of 100 μM Urumin and OVA (100% control) against PR8, H9N3 A/guinea fowl/Hong Kong/WF10/99, and a chimeric H9N3 including the WF10 head region and the PR8 conserved stalk.

FIG. 5A shows testing data of the ability of Alanine scanning mutants (FIG. 2A) of Urumin to inhibit PR8 virus. The reduction of viral titers by WT Urumin peptide is set as 0% and the increase or decrease in activity of each mutant peptide is compared to this baseline (peptide concentration 40 μM) by focus forming assay.

FIG. 5B shows comparison data of 100 μM L-vs. D-enantiomer of Urumin peptide against A/Puerto Rico/8/1934 influenza virus by focus forming assay.

FIG. 6A shows data on intranasal administration of Urumin peptide reduces influenza-induced morbidity, mortality, and lung viral titers in vivo. Morbidity and mortality assessment of Urumin treatment during 2×LD₅₀ live mouse adapted A/Puerto Rico/8/1934 influenza virus infection. Morbidity was assessed by percent of initial body mass over 14 days for OVA control and Urumin treated mice. If the body weights fell below 80% of original body mass, the mice were euthanized as per IACUC guidelines. Mortality was assessed by survival over 14 days.

FIG. 6B shows data on the effect of Urumin treatment on lung viral titers. Cohorts of BALB/c were treated with OVA or Urumin peptide and infected with 2×LD₅₀. At day 3 or 6 post-infection, animals were euthanized, lung tissue harvested and lung viral titers were compared between the two groups.

FIG. 7 shows data indicating Urumin is effective against drug-resistant influenza viruses. Testing of 100 μM Urumin or OVA peptide against neuraminidase inhibitor-resistant viruses. Four of these viruses, A/Louisiana/08/2013, A/Texas/23/2012, A/Chile/1579/2009, and A/District of Columbia/02/2014 are natural isolates. Five of these are reverse genetic viruses including NAI-sensitive controls (rgCa1/04/09/WT and rg1253(S)) and 3 resistant strains (rgCa1/04/09 H275Y, rgCa1/04/09 H275Y S247N, and rg1326(R)).

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature. It must be noted that, as used in the specification and the appended claims, the singular forms “a,” “an,” and “the” include plural referents unless the context clearly dictates otherwise.

The term “comprising” in reference to a peptide having an amino acid sequence refers a peptide that may contain additional N-terminal (amine end) or C-terminal (carboxylic acid end) amino acids, i.e., the term is intended to include the amino acid sequence within a larger peptide. The term “consisting of” in reference to a peptide having an amino acid sequence refers a peptide having the exact number of amino acids in the sequence and not more or having not more than a rage of amino acids expressly specified in the claim.

The influenza subtype “H1” refers influenza viruses that express a glycoprotein containing a haemagglutinin 1, traditionally the most common heamagglutinin found on the surface of infectious human influenza viruses. Real-Time RT-PCR methods can be used for in vitro qualitative detection and characterization of the human influenza virus RNA. Samples may be tested from in respiratory tract specimens of human patients presenting with signs and symptoms of respiratory infections. Commercial kits typically contain oligonucleotide primers, dual-labeled hydrolysis (TaqMan®) probes for the H1 subtype. The primers and/or probes typically target conserved regions of the hemagglutinin (HA) genes.

“Subject” refers any animal, preferably a human patient, livestock, or domestic pet.

The terms “protein” and “polypeptide” refer to compounds comprising amino acids joined via peptide bonds and are used interchangeably. Amino acids may be naturally or non-naturally occurring. A “chimeric protein” or “fusion protein” is a molecule in which different portions of the protein are derived from different origins such that the entire molecule is not naturally occurring. A chimeric protein may contain amino acid sequences from the same species of different species as long as they are not arranged together in the same way that they exist in a natural state. Examples of a chimeric protein include sequences disclosed herein that are contain one, two or more amino acids attached to the C-terminal or N-terminal end that are not identical to any naturally occurring protein, such as in the case of adding an amino acid containing an amine side chain group, e.g., lysine, an amino acid containing a carboxylic acid side chain group such as aspartic acid or glutamic acid, a polyhistidine tag, e.g. typically four or more histidine amino acids. Contemplated chimeric proteins include those with self-cleaving peptides such as P2A-GSG. See Wang. Scientific Reports 5, Article number: 16273 (2015).

A “variant” refers to a chemically similar sequence because of amino acid changes or chemical derivative thereof. In certain embodiments, a variant contains one, two, or more amino acid deletions or substitutions. In certain embodiments, the substitutions are conserved substitutions. In certain embodiments, a variant contains one, two, or ten or more an amino acid additions. The variant may be substituted with one or more chemical substituents.

One type of conservative amino acid substitutions refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. More rarely, a variant may have “non-conservative” changes (e.g., replacement of a glycine with a tryptophan). Similar minor variations may also include amino acid deletions or insertions (in other words, additions), or both. Guidance in determining which and how many amino acid residues may be substituted, inserted or deleted without abolishing biological activity may be found using computer programs well known in the art, for example, DNAStar software. Variants can be tested in functional assays. Certain variants have less than 10%, and preferably less than 5%, and still more preferably less than 2% changes (whether substitutions, deletions, and so on).

As used herein, the term “derivative” refers to a structurally similar peptide that retains sufficient functional attributes of the identified analogue. The derivative may be structurally similar because it is lacking one or more atoms, e.g., replacing an amino group, hydroxyl, or thiol group with a hydrogen, substituted, a salt, in different hydration/oxidation states, or because one or more atoms within the molecule are switched, such as, but not limited to, replacing a oxygen atom with a sulfur atom or replacing an amino group with a hydroxyl group. The derivative may be a prodrug, comprise a lipid, polyethylene glycol, saccharide, polysaccharide. A derivative may be two or more peptides linked together by a linking group. It is contemplated that the linking group may be biodegradable. Derivatives may be prepare by any variety of synthetic methods or appropriate adaptations presented in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

In certain embodiments, the peptides discloses herein have at least one non-naturally occurring molecular modification, such as the attachment of polyethylene glycol, the attachment of a chimeric peptide, the attachment of a fluorescent dye comprising aromatic groups, fluorescent peptide, a chelating agent capable of binding a radionuclide such as ¹⁸F, N-terminal acetyl, propionyl group, myristoyl and palmitoyl, group or N-terminal methylation, or a C-terminal alkyl ester. In certain embodiments, the disclosure contemplates the disclosure contemplates peptides disclosed herein labeled using commercially available biotinylation reagents. Biotinylated peptide can be used in streptavidin affinity binding, purification, and detection. In certain embodiments, the disclosure contemplates peptide disclose herein containing azide-derivatives of naturally occurring monosaccharides such as N-azidoacetylglucosamine, N-azidoacetylmannosamine, and N-azidoacetylgalactosamine.

In certain embodiments, this disclosure contemplates derivatives of peptide disclose herein wherein one or more amino acids are substituted with chemical groups to improve pharmacokinetic properties such as solubility and serum half-life, optionally connected through a linker. In certain embodiments, such a derivative may be a prodrug wherein the substituent or linker is biodegradable, or the substituent or linker is not biodegradable. In certain embodiments, contemplated substituents include a saccharide, polysaccharide, acetyl, fatty acid, lipid, and/or polyethylene glycol. The substituent may be covalently bonded through the formation of amide bonds on the C-terminus or N-terminus of the peptide optionally connected through a linker. In certain embodiments, it is contemplated that the substituent may be covalently bonded through an amino acid within the peptide, e.g. through an amine side chain group such as lysine or an amino acid containing a carboxylic acid side chain group such as aspartic acid or glutamic acid, within the peptide comprising a sequence disclosed herein. In certain embodiments, it is contemplated that the substituent may be covalently bonded through a cysteine in a sequence disclosed herein optionally connected through a linker. In certain embodiments, a substituent is connected through a linker that forms a disulfide with a cysteine amino acid side group.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRaRb, —NRaC(═O)Rb, —NRaC(═O)NRaNRb, —NRaC(═O)ORb, —NRaSO₂Rb, —C(═O)Ra, —C(═O)ORa, —C(═O)NRaRb, —OC(═O)NRaRb, —ORa, —SRa, —SORa, —S(═O)₂Ra, —OS(═O)₂Ra and —S(═O)₂ORa. Ra and Rb in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, and heteroarylalkyl. The substituents may further optionally be substituted.

As used herein, a “lipid” group refers to a hydrophobic group that is naturally or non-naturally occurring that is highly insoluble in water. As used herein a lipid group is considered highly insoluble in water when the point of connection on the lipid is replaced with a hydrogen and the resulting compound has a solubility of less than 0.63×10⁻⁴% w/w (at 25° C.) in water, which is the percent solubility of octane in water by weight. See Solvent Recovery Handbook, 2^{nd} Ed, Smallwood, 2002 by Blackwell Science, page 195. Examples of naturally occurring lipids include saturated or unsaturated hydrocarbon chains found in fatty acids, glycerolipids, cholesterol, steroids, polyketides, and derivatives. Non-naturally occurring lipids include derivatives of naturally occurring lipids, acrylic polymers, aromatic, and alkylated compounds and derivatives thereof.

The term “prodrug” refers to an agent that is converted into a biologically active form in vivo. Prodrugs are often useful because, in some situations, they may be easier to administer than the parent compound. The prodrug may also have improved solubility in pharmaceutical compositions over the parent drug. A prodrug may be converted into the parent drug by various mechanisms, including enzymatic processes and metabolic hydrolysis. Typical prodrugs are pharmaceutically acceptable esters. Prodrugs include compounds wherein a hydroxy, amino or mercapto (thiol) group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like.

For example, if a disclosed peptide or a pharmaceutically acceptable form of the peptide contains a carboxylic acid functional group, a prodrug can comprise a pharmaceutically acceptable ester formed by the replacement of the hydrogen atom of the acid group with a group such as (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as beta-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

If a disclosed peptide or a pharmaceutically acceptable form of the peptide contains an alcohol functional group, a prodrug can be formed by the replacement of the hydrogen atom of the alcohol group with a group such as (C₁-C₆)alkanoyloxymethyl, 1-((C₁-C₆)alkanoyloxy) ethyl, 1-methyl-1((C₁-C₆)alkanoyloxy)ethyl (C₁-C₆)alkoxycarbonyloxymethyl, —N—(C₁-C₆)alkoxycarbonylaminomethyl, succinoyl, (C₁-C₆)alkanoyl, alpha-amino(C₁-C₄)alkanoyl, arylacyl and alpha-aminoacyl, or alpha-aminoacyl-alpha-aminoacyl, where each alpha-aminoacyl group is independently selected from naturally occurring L-amino acids P(O)(OH)₂, —P(O)(O(C₁-C₆)alkyl)₂, and glycosyl (the radical resulting from the removal of a hydroxyl group of the hemiacetal form of a carbohydrate).

If a disclosed peptide or a pharmaceutically acceptable form of the peptide incorporates an amine functional group, a prodrug can be formed by the replacement of a hydrogen atom in the amine group with a group such as R-carbonyl, RO-carbonyl, NRR′-carbonyl where R and R′ are each independently (C₁-C₁₀)alkyl, (C₃-C₇)cycloalkyl, benzyl, a natural alpha-aminoacyl, —C(OH)C(O)OY₁ wherein Y¹ is H, (C₁-C₆)alkyl or benzyl, —C(OY₂)Y₃ wherein Y₂ is (C₁-C₄) alkyl and Y₃ is (C₁-C₆)alkyl, carboxy(C₁-C₆)alkyl, amino(C₁-C₄)alkyl or mono-Nor di-N,N—(C₁-C₆)alkylaminoalkyl, —C(Y₄)Y₅ wherein Y₄ is H or methyl and Y₅ is mono-N— or di-N,N—(C₁-C₆)alkylamino, morpholino, piperidin-1-yl or pyrrolidin-1-yl.

As used herein, “pharmaceutically acceptable esters” include, but are not limited to, alkyl, alkenyl, alkynyl, aryl, arylalkyl, and cycloalkyl esters of acidic groups, including, but not limited to, carboxylic acids, phosphoric acids, phosphinic acids, sulfonic acids, sulfinic acids, and boronic acids.

As used herein, “pharmaceutically acceptable enol ethers” include, but are not limited to, derivatives of formula —C═C(OR) where R can be selected from alkyl, alkenyl, alkynyl, aryl, aralkyl, and cycloalkyl. Pharmaceutically acceptable enol esters include, but are not limited to, derivatives of formula —C═C(OC(O)R) where R can be selected from hydrogen, alkyl, alkenyl, alkynyl, aryl, aralkyl, and cycloalkyl.

A “linking group” refers to any variety of molecular arrangements that can be used to bridge to molecular moieties together. An example formula may be —Rₘ— wherein R is selected individually and independently at each occurrence as: —CRₘRₘ—, —CHRₘ—, —CH—, —C—, —CH₂—, —C(OH)Rₘ, —C(OH)(OH)—, —C(OH)H, —C(Hal)Rₘ—, —C(Hal)(Hal)-, —C(Hal)H—, —C(N3)Rₘ—, —C(CN)Rₘ—, —C(CN)(CN)—, —C(CN)H—, —C(N₃)(N₃)—, —C(N₃)H—, —O—, —S—, —N—, —NH—, —NRₘ—, —(C═O)—, —(C═NH)—, —(C═S)—, —(C═CH₂)—, which may contain single, double, or triple bonds individually and independently between the R groups. If an R is branched with an Rₘ it may be terminated with a group such as —CH₃, —H, —CH═CH₂, —CCH, —OH, —SH, —NH₂, —N₃, —CN, or -Hal, or two branched Rs may form a cyclic structure. It is contemplated that in certain instances, the total Rs or “m” may be less than 100, or 50, or 25, or 10. Examples of linking groups include bridging alkyl groups and alkoxyalkyl groups. Linking groups may be substituted with one or more substituents.

As used herein, the term “biodegradable” in reference to a substituent or linker refers to a molecular arrangement in a peptide derivative that when administered to a subject, e.g., human, will be broken down by biological mechanism such that a metabolite will be formed and the molecular arrangement will not persist for over a long period of time, e.g., the molecular arrangement will be broken down by the body after a several hours or days. In certain embodiments, the disclosure contemplates that the biodegradable linker or substituent will not exist after a week or a month.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g. patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

As used herein, the term “sterilized” refers to subjecting something to a process that effectively kills or eliminates transmissible agents (such as fungi, bacteria, viruses, prions and spore forms etc.). Sterilization can be achieved through application of heat, chemicals, irradiation, high pressure or filtration. One process involves water prepared by distillation and stored in an airtight container wherein suitable additives are introduced to approximate isotonicity.

The term “polynucleotide” refers to a molecule comprised of two or more deoxyribonucleotides or ribonucleotides, preferably more than three, and usually more than ten. The exact size will depend on many factors, which in turn depends on the ultimate function or use of the oligonucleotide. The polynucleotide may be generated in any manner, including chemical synthesis, DNA replication, reverse transcription, or a combination thereof. The term “oligonucleotide” generally refers to a short length of single-stranded polynucleotide chain usually less than 30 nucleotides long, although it may also be used interchangeably with the term “polynucleotide.”

The term “nucleic acid” refers to a polymer of nucleotides, or a polynucleotide, as described above. The term is used to designate a single molecule, or a collection of molecules. Nucleic acids may be single stranded or double stranded, and may include coding regions and regions of various control elements.

A “heterologous” nucleic acid sequence or peptide sequence refers to a nucleic acid sequence or peptide sequence that do not naturally occur, e.g., because the whole sequences contain a segment from other plants, bacteria, viruses, other organisms, or joinder of two sequences that occur the same organism but are joined together in a manner that does not naturally occur in the same organism or any natural state.

The term “recombinant” when made in reference to a nucleic acid molecule refers to a nucleic acid molecule which is comprised of segments of nucleic acid joined together by means of molecular biological techniques provided that the entire nucleic acid sequence does not occurring in nature, i.e., there is at least one mutation in the overall sequence such that the entire sequence is not naturally occurring even though separately segments may occurring in nature. The segments may be joined in an altered arrangement such that the entire nucleic acid sequence from start to finish does not naturally occur. The term “recombinant” when made in reference to a protein or a polypeptide refers to a protein molecule that is expressed using a recombinant nucleic acid molecule.

The terms “vector” or “expression vector” refer to a recombinant nucleic acid containing a desired coding sequence and appropriate nucleic acid sequences necessary for the expression of the operably linked coding sequence in a particular host organism or expression system, e.g., cellular or cell-free. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

Protein “expression systems” refer to in vivo and in vitro (cell free) systems. Systems for recombinant protein expression typically utilize cells transfecting with a DNA expression vector that contains the template. The cells are cultured under conditions such that they translate the desired protein. Expressed proteins are extracted for subsequent purification. In vivo protein expression systems using prokaryotic and eukaryotic cells are well known. Proteins may be recovered using denaturants and protein-refolding procedures. In vitro (cell-free) protein expression systems typically use translation-compatible extracts of whole cells or compositions that contain components sufficient for transcription, translation and optionally post-translational modifications such as RNA polymerase, regulatory protein factors, transcription factors, ribosomes, tRNA cofactors, amino acids and nucleotides. In the presence of an expression vectors, these extracts and components can synthesize proteins of interest. Cell-free systems typically do not contain proteases and enable labeling of the protein with modified amino acids. Some cell free systems incorporated encoded components for translation into the expression vector. See, e.g., Shimizu et al., Cell-free translation reconstituted with purified components, 2001, Nat. Biotechnol., 19, 751-755 and Asahara & Chong, Nucleic Acids Research, 2010, 38(13): e141, both hereby incorporated by reference in their entirety.

A “selectable marker” is a nucleic acid introduced into a recombinant vector that encodes a polypeptide that confers a trait suitable for artificial selection or identification (report gene), e.g., beta-lactamase confers antibiotic resistance, which allows an organism expressing beta-lactamase to survive in the presence antibiotic in a growth medium. Another example is thymidine kinase, which makes the host sensitive to ganciclovir selection. It may be a screenable marker that allows one to distinguish between wanted and unwanted cells based on the presence or absence of an expected color. For example, the lac-z-gene produces a beta-galactosidase enzyme that confers a blue color in the presence of X-gal (5-bromo-4-chloro-3-indolyl-β-D-galactoside). If recombinant insertion inactivates the lac-z-gene, then the resulting colonies are colorless. There may be one or more selectable markers, e.g., an enzyme that can complement to the inability of an expression organism to synthesize a particular compound required for its growth (auxotrophic) and one able to convert a compound to another that is toxic for growth. URA3, an orotidine-5′ phosphate decarboxylase, is necessary for uracil biosynthesis and can complement ura3 mutants that are auxotrophic for uracil. URA3 also converts 5-fluoroorotic acid into the toxic compound 5-fluorouracil. Additional contemplated selectable markers include any genes that impart antibacterial resistance or express a fluorescent protein. Examples include, but are not limited to, the following genes: amp^{r}, cam^{r}, tet^{r}, blasticidin^{r}, neo^{r}, hyg^{r}, abx^{r}, neomycin phosphotransferase type II gene (nptII), p-glucuronidase (gus), green fluorescent protein (gfp), egfp, yfp, mCherry, p-galactosidase (lacZ), lacZa, lacZAM15, chloramphenicol acetyltransferase (cat), alkaline phosphatase (phoA), bacterial luciferase (luxAB), bialaphos resistance gene (bar), phosphomannose isomerase (pmi), xylose isomerase (xylA), arabitol dehydrogenase (atlD), UDP-glucose:galactose-1-phosphate uridyltransferasel (galT), feedback-insensitive a subunit of anthranilate synthase (OASA1D), 2-deoxyglucose (2-DOGR), benzyladenine-N-3-glucuronide, E. coli threonine deaminase, glutamate 1-semialdehyde aminotransferase (GSA-AT), D-amino acidoxidase (DAAO), salt-tolerance gene (rstB), ferredoxin-like protein (pflp), trehalose-6-P synthase gene (AtTPS1), lysine racemase (lyr), dihydrodipicolinate synthase (dapA), tryptophan synthase beta 1 (AtTSB 1), dehalogenase (dhlA), mannose-6-phosphate reductase gene (M6PR), hygromycin phosphotransferase (HPT), and D-serine ammonialyase (dsdA).

A “label” refers to a detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes. In one example, a “label receptor” refers to incorporation of a heterologous polypeptide in the receptor. A label includes the incorporation of a radiolabeled amino acid or the covalent attachment of biotinyl moieties to a polypeptide that can be detected by marked avidin (for example, streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: radioisotopes or radionucleotides (such as ³⁵S or ¹³¹I) fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some embodiments, labels are attached by spacer arms of various lengths to reduce potential steric hindrance.

In certain embodiments, the disclosure relates to recombinant polypeptides comprising sequences disclosed herein or variants or fusions thereof wherein the amino terminal end or the carbon terminal end of the amino acid sequence are optionally attached to a heterologous amino acid sequence, label, or reporter molecule.

In certain embodiments, the disclosure relates to the recombinant vectors comprising a nucleic acid encoding a polypeptide disclosed herein or chimeric protein thereof.

In certain embodiments, the recombinant vector optionally comprises a mammalian, human, insect, viral, bacterial, bacterial plasmid, yeast associated origin of replication or gene such as a gene or retroviral gene or lentiviral LTR, TAR, RRE, PE, SLIP, CRS, and INS nucleotide segment or gene selected from tat, rev, nef, vif, vpr, vpu, and vpx or structural genes selected from gag, pol, and env.

In certain embodiments, the recombinant vector optionally comprises a gene vector element (nucleic acid) such as a selectable marker region, lac operon, a CMV promoter, a hybrid chicken B-actin/CMV enhancer (CAG) promoter, tac promoter, T7 RNA polymerase promoter, SP6 RNA polymerase promoter, SV40 promoter, internal ribosome entry site (IRES) sequence, cis-acting woodchuck post regulatory regulatory element (WPRE), scaffold-attachment region (SAR), inverted terminal repeats (ITR), FLAG tag coding region, c-myc tag coding region, metal affinity tag coding region, streptavidin binding peptide tag coding region, polyHis tag coding region, HA tag coding region, MBP tag coding region, GST tag coding region, polyadenylation coding region, SV40 polyadenylation signal, SV40 origin of replication, Col E1 origin of replication, f1 origin, pBR322 origin, or pUC origin, TEV protease recognition site, loxP site, Cre recombinase coding region, or a multiple cloning site such as having 5, 6, or 7 or more restriction sites within a continuous segment of less than 50 or 60 nucleotides or having 3 or 4 or more restriction sites with a continuous segment of less than 20 or 30 nucleotides.

In certain embodiments, term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity.

In certain embodiments, sequence “identity” refers to the number of exactly matching amino acids (expressed as a percentage) in a sequence alignment between two sequences of the alignment calculated using the number of identical positions divided by the greater of the shortest sequence or the number of equivalent positions excluding overhangs wherein internal gaps are counted as an equivalent position. For example, the polypeptides GGGGGG and GGGGT have a sequence identity of 4 out of 5 or 80%. For example, the polypeptides GGGPPP and GGGAPPP have a sequence identity of 6 out of 7 or 85%. In certain embodiments, any recitation of sequence identity expressed herein may be substituted for sequence similarity. Percent “similarity” is used to quantify the similarity between two sequences of the alignment. This method is identical to determining the identity except that certain amino acids do not have to be identical to have a match. Amino acids are classified as matches if they are among a group with similar properties according to the following amino acid groups: Aromatic—F Y W; hydrophobic—A V I L; Charged positive: R K H; Charged negative—D E; Polar—S T N Q.

As used herein, the term “combination with” when used to describe administration with an additional treatment means that the agent may be administered prior to, together with, or after the additional treatment, or a combination thereof.

### Pharmaceutical Methods and Compositions

Methods of administering peptides include, but are not limited to, parenteral administration (e.g., intradermal, intramuscular, intraperitoneal, intravenous and subcutaneous), epidural, and mucosal (e.g., intranasal and oral routes). In a specific embodiment, the peptides or chimeric proteins are administered intramuscularly, intravenously, or subcutaneously. The compositions may be administered by any convenient route, for example, by infusion or bolus injection, by absorption through epithelial or mucocutaneous linings (e.g., oral mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent. See, e.g., U.S. Pat. Nos. 6,019,968; 5,985,20; 5,985,309; 5,934,272; 5,874,064; 5,855,913; 5,290,540; and 4,880,078; and PCT Publication Nos. WO 92/19244; WO 97/32572; WO 97/44013; WO 98/31346; and WO 99/66903. In a specific embodiment, it may be desirable to administer the pharmaceutical compositions locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers. In certain embodiments, the aerosolizing agent or propellant is a hydrofluoroalkane, 1,1,1,2-tetrafluoroethane, 1,1,1,2,3,3,3-heptafluoropropane, propane, n-butane, isobutene, carbon dioxide, air, nitrogen, nitrous oxide, dimethyl ether, trans-1,3,3,3-tetrafluoroprop-1-ene, or combinations thereof.

The precise dose to be employed in the formulation will also depend on the route of administration, and the seriousness of the condition, and should be decided according to the judgment of the practitioner and each patient's circumstances. Effective doses may be extrapolated from dose-response curves derived from in vitro or animal model test systems. For peptides and fusion proteins, the dosage administered to a patient is typically 0.0001 mg/kg to 100 mg/kg of the patient's body weight. Preferably, the dosage administered to a patient is between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight. Further, the dosage and frequency of administration of proteins may be reduced by enhancing uptake and tissue penetration of the fusion proteins by modifications such as, for example, lipidation.

The compositions include bulk drug compositions useful in the manufacture of pharmaceutical compositions (e.g., impure or non-sterile compositions) and pharmaceutical compositions (i.e., compositions that are suitable for administration to a subject or patient) which can be used in the preparation of unit dosage forms. Such compositions comprise a prophylactically or therapeutically effective amount of a prophylactic and/or therapeutic agent disclosed herein or a combination of those agents and a pharmaceutically acceptable carrier. In certain embodiments, the pharmaceutical compositions contain a pharmaceutically acceptable excipient that is a solubilizing agent such as a lipid, cholesterol, fatty acid, fatty acid alkyl ester, linoleic acid, oleic acid arachidonic acid, saccharide, polysaccharide, cyclodextrin, 2-hydoxypropyl(cyclodextrin), or combinations thereof.

In certain embodiments, the pharmaceutical compositions is in solid form surrounded by an enteric coating, i.e., a polymer barrier applied on oral medication that prevents its dissolution or disintegration in the gastric environment. Compounds typically found in enteric coatings include methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, and combinations thereof.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions can be formulated as neutral or salt forms. Pharmaceutically acceptable salts include, but are not limited to, those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

One embodiment provides a pharmaceutical pack or kit comprising one or more containers filled with peptides disclosed herein. Additionally, one or more other prophylactic or therapeutic agents useful for the treatment of a disease can also be included in the pharmaceutical pack or kit. One embodiment provides a pharmaceutical pack or kit including one or more containers filled with one or more of the ingredients of the pharmaceutical compositions. Optionally associated with such container(s) can be a notice in the form prescribed by a governmental agency regulating the manufacture, use or sale of pharmaceuticals or biological products, which notice reflects approval by the agency of manufacture, use or sale for human administration.

In certain embodiment, this disclosure contemplates pharmaceutical compositions comprising proteins disclosed herein and pharmaceutically acceptable excipient. In certain embodiments, this disclosure contemplates the production of a medicament comprising proteins disclosed herein and uses for methods disclosed herein.

In certain embodiments, the disclosure relates to pharmaceutical compositions comprising proteins disclosed herein and a pharmaceutically acceptable excipient. In certain embodiments, the composition is a pill or in a capsule or the composition is an aqueous buffer, e.g., a pH between 6 and 8. In certain embodiments, the pharmaceutically acceptable excipient is selected from a filler, glidant, binder, disintegrant, lubricant, and saccharide.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents solvents or vehicles include water, ethanol, polyols (propylene glycol, polyethylene glycol, glycerol, and the like), suitable mixtures thereof, vegetable (such as olive oil, sesame oil and viscoleo) and injectable organic esters such as ethyl oleate.

Prevention of the action of microorganisms may be controlled by addition of any of various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

Solid dosage forms for oral administration include capsules, tablets, pills, powders and granules. In such solid dosage forms, the proteins may be admixed with at least one inert customary excipient (or carrier) such as sodium citrate or dicalcium phosphate or: (a) fillers or extenders, as for example, starches, lactose, sucrose, glucose, mannitol and silicic acid, (b) binders, as for example, carboxymethylcellulose, alginates, gelatin, polyvinylpyrrolidone, sucrose, and acacia, (c) humectants, as for example, glycerol (d) disintegrating agents, as for example, agar-agar, calcium carbonate, potato or tapioca starch, alginic acid, certain complex silicates, and sodium carbonate, (e) solution retarders, as for example paraffin, (f) absorption accelerators, as for example, quaternary ammonium compounds, (g) wetting agents, as for example cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Liquid dosage forms for oral administration include pharmaceutically acceptable emulsions, solutions, suspensions, syrups, and elixirs. In addition to the proteins, the liquid dosage forms may contain inert diluents commonly used in the art, such as water or other solvents, solubilizing agents and emulsifiers, for example, ethyl alcohol, isopropyl alcohol, ethyl carbonate, ethyl acetate, benzyl alcohol, benzyl benzoate, propylene glycol, 1,3-butylene glycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like.

In certain embodiments, production processes are contemplated which two components, proteins disclosed herein and a pharmaceutical carrier, are provided already in a combined dry form ready to be reconstituted together. In other embodiments, it is contemplated that proteins disclosed herein and a pharmaceutical carrier are admixed to provide a pharmaceutical composition.

Providing a pharmaceutic composition is possible in a one-step process, simply by adding a suitable pharmaceutically acceptable diluent to the composition in a container. In certain embodiments, the container is preferably a syringe for administering the reconstituted pharmaceutical composition after contact with the diluent. In certain embodiments, the coated proteins can be filled into a syringe, and the syringe can then be closed with the stopper. A diluent is used in an amount to achieve the desired end-concentration. The pharmaceutical composition may contain other useful component, such as ions, buffers, excipients, stabilizers, etc.

A “dry” pharmaceutical composition typically has only a residual content of moisture, which may approximately correspond to the moisture content of comparable commercial products, for example, has about 12% moisture as a dry product. Usually, the dry pharmaceutical composition according to the present invention has a residual moisture content preferably below 10% moisture, more preferred below 5% moisture, especially below 1% moisture. The pharmaceutical composition can also have lower moisture content, e.g. 0.1% or even below. In certain embodiments, the pharmaceutical composition is provided in dry in order to prevent degradation and enable storage stability.

A container can be any container suitable for housing (and storing) pharmaceutically compositions such as syringes, vials, tubes, etc. The pharmaceutical composition may then preferably be applied via specific needles of the syringe or via suitable catheters. A typical diluent comprises water for injection, and NaCl (preferably 50 to 150 mM, especially 110 mM), CaCl₂ (preferably 10 to 80 mM, especially 40 mM), sodium acetate (preferably 0 to 50 mM, especially 20 mM) and mannitol (preferably up to 10% w/w, especially 2% w/w). Preferably, the diluent can also include a buffer or buffer system so as to buffer the pH of the reconstituted dry composition, preferably at a pH of 6.2 to 7.5, especially at pH of 6.9 to 7.1.

In certain embodiments, the diluent is provided in a separate container. This can preferably be a syringe. The diluent in the syringe can then easily be applied to the container for reconstitution of the dry compositions. If the container is also a syringe, both syringes can be finished together in a pack. It is therefore preferred to provide the dry compositions in a syringe, which is finished with a diluent syringe with a pharmaceutically acceptable diluent for reconstituting, said dry and stable composition.

In certain embodiments, this disclosure contemplates a kit comprising a pharmaceutical composition disclosed herein and a container with a suitable diluent. Further components of the kit may be instructions for use, administration means, such as syringes, catheters, brushes, etc. (if the compositions are not already provided in the administration means) or other components necessary for use in medical (surgical) practice, such as substitute needles or catheters, extra vials or further wound cover means. In certain embodiments, the kit comprises a syringe housing the dry and stable hemostatic composition and a syringe containing the diluent (or provided to take up the diluent from another diluent container).

### Examples

### Skin Secretion Harvesting

Adult specimens of H. bahuvistara (both sexes, n=15) were collected from the Northern part (Kanhangad) of Kerala, India, under the license from Kerala Forest Department, India during 2012. Skin secretion from each specimen was collected by giving a mild transdermal electrical stimulation (6vDC, 4 ms pulse width, 50HZ) for 20s duration. During the electrical stimulation, the skin was rinsed with Milli-Q water and the aqueous solution was collected and immediately fixed in liquid nitrogen, brought back to the laboratory, lyophilized and stored at −80° C. prior to analysis. The frogs were released in a healthy state back to the habitat from which it was collected. No adverse events were noticed in the specimens after stimulation.

Molecular Cloning of cDNAs Encoding Antimicrobial Peptides

Poly(A) mRNAs were isolated from the lyophilized secretion using Dyna Beads® (Dynal Biotech, UK) in accordance with manufacturer's instructions. cDNA library was constructed using SMARTerTMcDNA Amplification Kit (Clontech, UK) in agreement with manufacturer's instructions. SMART MMLV RT and the primers, SMARTer II A Oligonucleotide Primer 5′-AAGCAGTGGTATCAACGCAGAGTACGCGGG-3′ (SEQ ID NO: 28) and 3′CDS Primer A 5′-AAGCAGTGGTATCAACGCAGAGTAC (T) 30VN— 3′ (SEQ ID NO: 29) (N=A, C, G or T; V=A, G, C) were used to synthesize the first-strand cDNA. Advantage DNA Polymerase was used to amplify the second strand by the primers 3′CDS Primer A and 5′ PCR primer 5′-AAGCAGTGGTATCAACGCAGAGT-3′ (SEQ ID NO: 30).

Screening of cDNAs encoding antimicrobial peptides was carried out with two sense primers, including a specific primer designed for ranid frogs from the nucleotide sequence of the highly conserved signal peptide region and 5′-untranslated region of antimicrobial peptide-encoding cDNAs and a degenerate primer. 3′CDS primer A was used as the anti-sense primer.

Advantage DNA Polymerase (Clontech, UK) was used for PCR with the following conditions: 94° C. for 2 minutes; followed by 30 cycles of 92° C. for 10s, 50° C. for 30s, 72° C. for 40s; and again, followed by a final extension at 72° C. for 10 minutes. Gel purified PCR products were cloned into pGEM-T easy vector system (Promega Corp.) followed by plasmid isolation. Purified plasmids were sequenced using ABI 3730 automated sequencer. Nucleotide sequences obtained were translated. The peptide sequence obtained was subjected to homology searches using BLAST (NCBI) to confirm their identity.

Peptides were synthesized at purity greater than 80% without specific disulfide bridge modifications. As control, OVA257-264 peptide (InvivoGen) was used.

### Peptide Administration and Live Virus Challenge

Cohorts of age- and sex-matched BALB/c mice were anesthetized and administered intranasally 20 μg Urumin or OVA peptide in 20 μl, and then five minutes later infected intranasally with 2×LD50 of mouse adapted PR8 in PBS (30 μl volume). On each of the following three days, mice were anesthetized and intranasally given 20 μg of Urumin or OVA peptide in 20 μl. For lung viral titer assessment, cohorts of mice were administered intranasally with 60 μg, 20 μg, or 6.6 μg doses of Urumin or OVA peptide (20 μl volume) and infected with 2×LD₅₀ mouse adapted PR8 influenza virus five minutes later. On each of the following 3 days, mice were intranasally given 60 μg, 20 μg, or 6.6 μg doses of Urumin or OVA peptide. At days 3 and 6, mice were euthanized and their lungs processed for viral titers.

A peptide from hydrophylax bahuvistara exhibits anti-A/PR/8/1934 influenza virus activity in vitro Cohorts of the Indian frog, Hydrophylax bahuvistara, were captured. Skin secretions were collected from them via mild electrical stimulation and then returned the frogs to their natural habitat, unharmed. Host defense peptides were identified from these secretions. The peptides were synthesized for screening. These peptides were screened for potential activity against the human H1N1 influenza A virus A/Puerto Rico/8/1934 (PR8) using a plaque reduction assay. Briefly, frog peptides or control OVA peptide were incubated individual with the virus for 2 hours and plated with MDCK cells. Virus plaque reduction was enumerated 3 days later.

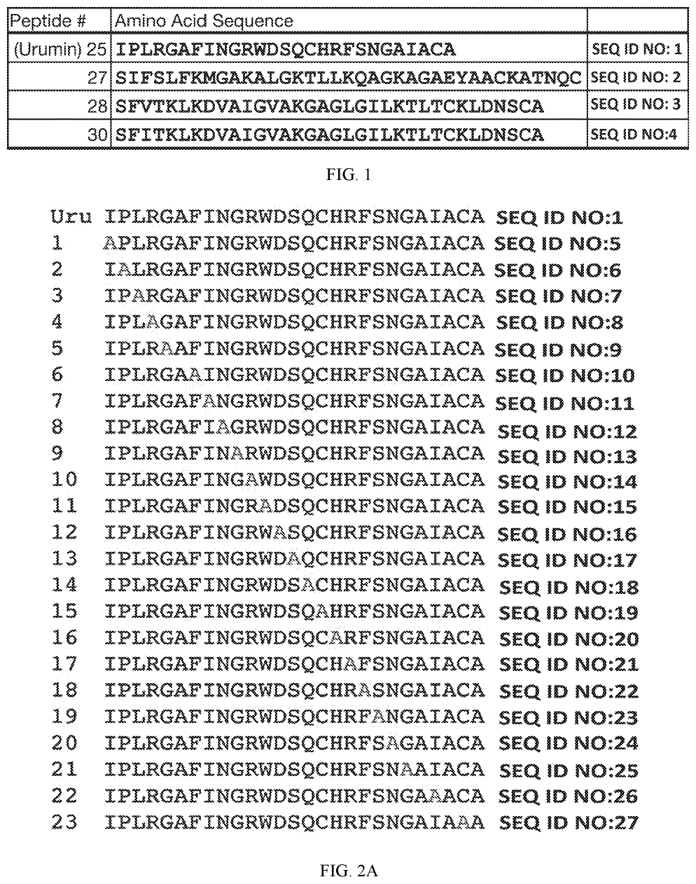

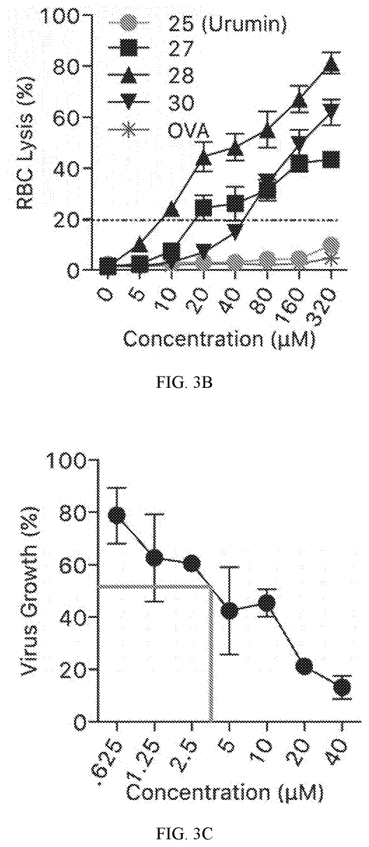

Peptides were identified that showed greater than 50% reduction of viral titers as compared to the control OVA peptide (FIG. 1). Since host defense peptides are often toxic to mammalian cells, especially at higher concentrations, the toxicity of these peptides were measured by incubating them with human erythrocytes for 1 hour. Hemolysis was evaluated. Peptide #25, showed no toxicity up to 320 μM in PBS (FIG. 3B). This peptide reached a 20% level of toxicity at a concentration of 1430 μM. This peptide was named Urumin from the word “urumi”, which is a deadly whip sword that originated from the same geographical region as H. bahuvistara. Urumin is a 27 amino acid peptide with no known homology to any other host defense peptide (FIG. 1).

The dose responsiveness and kinetics of its antiviral activity was evaluated. The IC₅₀ of Urumin is 3.8 μM (FIG. 3C) and increasing the peptide concentration led to significantly higher (p=0.0004) virus inhibition. The therapeutic index (TI) or safety window of a host defense agent compares the therapeutic effect to the toxicity. For Urumin, the toxic dose (TD50) is 2450 PM and IC50 is 3.8 μM (FIG. 1E). TD50/IC50 produces a TI of 644.7, where the greater the number is than 1, the more favorable the safety of the therapeutic is. Additionally, the antiviral activity occurs within 15 minutes of incubation with the peptide. Urumin had no antiviral effect on Human Immunodeficiency virus, Simian immunodeficiency virus, Herpes Simplex Virus II, Hepatitis C, Ebola, Zika and Dengue viruses.

### H. Bahuvistara Peptide Urumin is Specific for H1 Hemagglutinin and Targets the Conserved Stalk Region of H1 HA

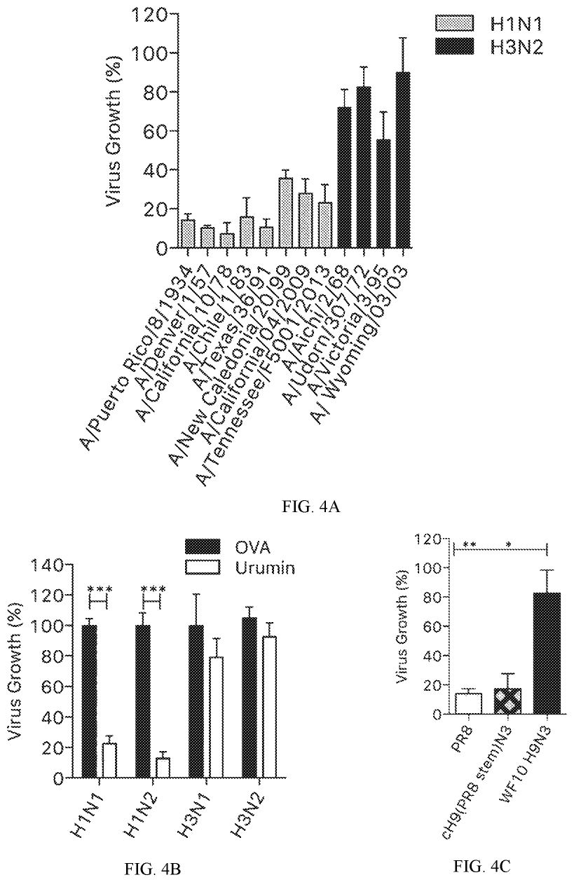

Since Urumin specifically targeted PR8H1N1 influenza virus, whether this antiviral effect was strain or subtype specific was examined. Briefly, Urumin or control OVA peptide were incubated with the virus for 2 hours, followed by infection of MDCK cells. Virus plaques were enumeration of 3 days later. Urumin was tested against 8 different H1N1 influenza strains and 4 different H3N2 influenza viruses. These viruses were circulating strains from 1934-2013 and included the 2009 pandemic strain (A/California/04/2009) and a 2013 drift variant of this virus (A/Tennessee/F5001/2013). Of the 12 viruses, Urumin peptide inhibited all 8H1N1 viruses by at least 60%, with titers of some strains decreasing by over 90%. In contrast, all 4 of the H3N2 viruses were reduced by less than 50%, with 3 of 4 being reduced by less than 30% (FIG. 4A). Overall, Urumin has a significantly greater effect against H1N1 strains than H3N2 influenza strains. These results suggest that Urumin could be targeting the surface H1 HA, N1 NA, or potentially both. To more precisely determine the specificity of Urumin peptide, reassortant PR8 viruses that bear the same six internal gene segments but differ in their HA and NA expression were used. Four PR8 reassortant viruses—H1N1, H1N2, H3N1, and H3N2 were tested against Urumin or the control OVA peptide. Of these viruses, Urumin treatment significantly reduced titers of H1N1 and H1N2 viruses, while the H3N1 and H3N2 were unaffected, demonstrating that the Urumin peptide inhibits influenza viruses that bear H1 HA but not H3 HA, or N1/N2 NA.

The HA protein is a trimer that consists of a conserved stalk region and a more variable globular head region. Targeting the conserved stalk region of HA, as in broadly neutralizing antibodies or computationally designed stalk binding proteins, present a promising approach to neutralizing human influenza viruses. Since Urumin peptide targets H1 HA, and inhibited 8H1N1 viruses that circulated over a span of 75 years, it was reasoned that it targets the conserved stalk region and not the variable globular head region. In order to test this, the H9N3 virus, A/guinea fowl/Hong Kong/WF10/1999 and a chimeric form of this virus, where the HA consists of the H9 head region, and the PR8H1 stalk were utilized. Urumin peptide inhibited the chimeric virus with the H1 stalk and H9 head and produced a similar level of reduction as observed with PR8. In contrast, the non-chimeric wild-type H9N3 virus (H9 stalk and H9 head) was not inhibited suggesting that the Urumin peptide specifically targeted the stalk region of H1 HA.

Whether Urumin would block the binding of monoclonal antibodies directed to the stalk but not head region of HA was evaluated. A competitive ELISA was conducted where recombinant purified HA was incubated with serially diluted Urumin or OVA peptide and assessed the ability to block the binding of monoclonal antibodies directed at the head or stalk regions of HA. Urumin peptide inhibited the binding of the anti-stalk but not anti-head monoclonal antibodies (FIG. 4C). Taken together, these data demonstrate that the Urumin peptide binds to the conserved stalk region of H1 HA to inhibit influenza viruses.

### Urumin Disrupts Influenza Virus Integrity

Having demonstrated that Urumin effectively inhibits influenza viruses, whether the peptide actively destroys influenza viral particles or whether it inhibits viral growth was evaluated. PR8 virus was incubated with Urumin or OVA peptides for 2 hours and then imaged by electron microscopy. OVA peptide-treated virus particles appear intact, while Urumin peptide treated influenza virions were completely destroyed.

### Urumin Requires Sequence Fidelity and Chirality for Activity

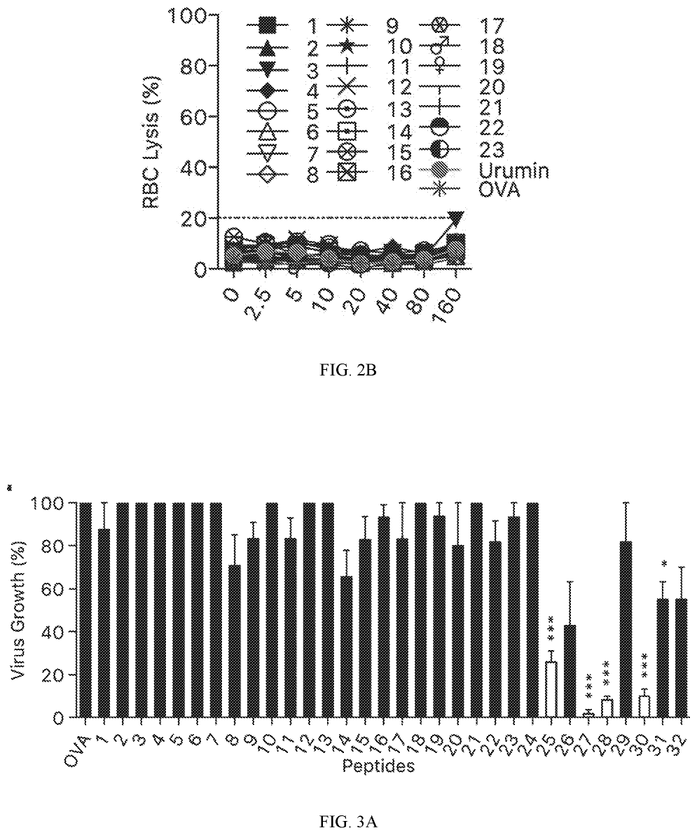

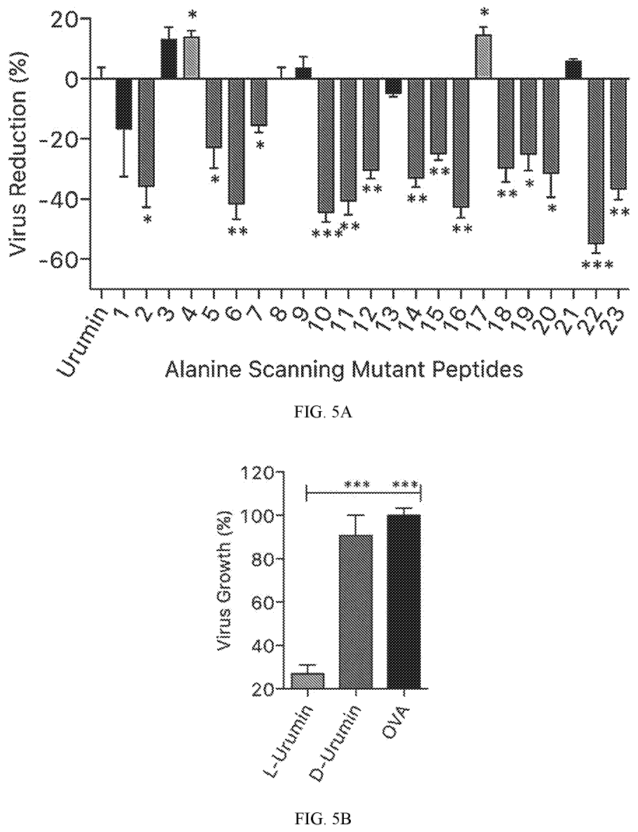

Which amino acid residues in Urumin peptide were important for its activity against H1 influenza viruses was evaluated. Alanine scan mutant were generated. (FIG. 2A). Whether these mutations increased toxicity to human red blood cells was tested. None of the alanine mutants showed toxicity to erythrocytes, with the exception of a mutation at the 3rd residue, where we observed 20% lysis at the high concentration of 160 μM. To test the mutants for anti-influenza activity, a focus-forming assay was utilized, which allowed us to move from 6-12-well plates to a 96-well format. MDCK-SIAT-I cells, which over express α2,6-linked sialic acid, was infected. Of the 23 mutants, 13 (57%) showed less antiviral activity than Urumin, 10 (43%) had equivalent activity to Urumin, and no mutations significantly improved peptide-induced viral reduction (FIG. 5A). Whether peptide chirality had an impact on the effectiveness of Urumin anti-influenza activity was tested. To conduct this experiment, a stereoisomer variant of Urumin was produced using D rather than L amino acids. This D amino acid Urumin variant, like the L variant, elicited no toxicity in human red blood cells. Anti-PR8 activity of D-Urumin in comparison to L-Urumin as well as OVA control peptide was assessed. In contrast to L-Urumin, D-Urumin showed no reduction of the PR8 influenza virus, demonstrating that Urumin recognizes a target with a chiral center.

### Intranasal Administration of Urumin Reduces Influenza-Induced Morbidity, Mortality, and Lung Viral Titers In Vivo

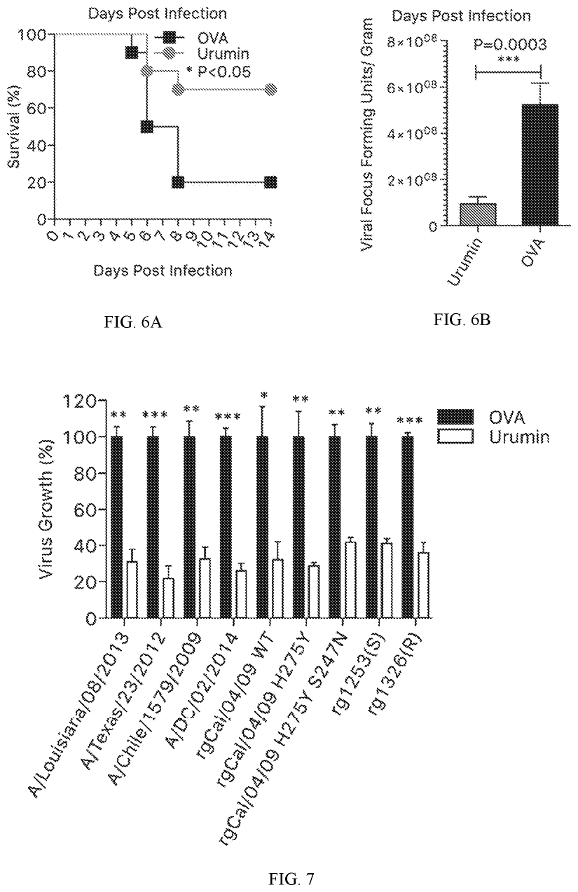

Whether Urumin could exert its antiviral activity in vivo by testing the extent to which peptide treatment could protect naïve mice infected with live mouse adapted H1N1 PR8 virus was investigated. Cohorts of BALB/c mice were treated with 20 μg Urumin or an OVA peptide control, administered intranasally. Five minutes later, they were infected intranasally with 2×LD₅₀ of live mouse-adapted PR8 influenza virus. The mice were then treated daily for the next 3 days with 20 μg of Urumin or OVA peptide intranasally. Ideally, the peptide should be delivered systemically but we chose the suboptimal intranasal route for this proof of principle confirmation that Urumin is functional in vivo because strategies to deliver the peptide systemically need to be worked out. Morbidity (percent mass reduction) and mortality (survival) were assessed over 14 days following infection. There was a significant difference in morbidity as measured by percent body mass loss between control and Urumin treated groups wherein the Urumin treated mice fared much better. Furthermore, 70% of Urumin peptide-treated mice survived 2×LD₅₀ influenza infection while only 20% of OVA-treated control mice survived (FIG. 6A).

In a separate experiment, the extent to which intranasal administration of Urumin peptide reduced lung virus titers in infected mice was determined. Cohorts of BALB/c mice were administered with 6.6 μg, 20 μg, or 60 μg of Urumin or OVA peptide and infected with 2×LD₅₀ of live mouse-adapted PR8 influenza virus in the same manner as the morbidity and mortality analysis described above. The animals were euthanized at days 3 and 6, and lung viral titers were assessed by focus-forming assay. There is a significant (80% with 6.6 μg. 82% with 20 μg, and 92% with 60 μg) decrease in viral focus-forming units per gram of lung tissue in the Urumin peptide treated cohort as compared to control mice at day 6 (FIG. 6B).

### Urumin is Effective Against Drug-Resistant Influenza Viruses

Since Urumin targets HA and not NA or M2, whether Urumin is effective at neutralizing drug-resistant H1N1 viruses was evaluated. Current anti-influenza virus drugs target NA and M2 proteins and drug resistance is common. Urumin was tested against 7 drug-resistant influenza strains. Four of these viruses, acquired from the CDC, are resistant to oseltamivir carboxylate (A/Louisiana/08/2013 H275Y, A/Texas/23/2012 H275Y), zanamivir and peramivir (A/District of Columbia/02/2014 C11-7), or oseltamivir carboxylate and zanamivir (A/Chile/1579/2009 1223K). Five drug resistant viruses generated by reverse genetics were also tested. These included a control drug sensitive pandemic H1N1 rgCal/04/09 wild-type, as well as oseltamivir-resistant mutants rgCal/04/09 H275Y and rgCal/04/09 H275Y S247N, an oseltamivir-sensitive A/New York/08-1253/2008 (rg1253 (S)) and oseltamivir-resistant A/New York/08-1326/2008 (rg1326(R)). All of these drug-resistant and control influenza viruses were tested against Urumin or an OVA control peptide and our data shows that Urumin significantly reduced the viral titers of all drug-resistant and drug sensitive H1N1 virus strains assessed (FIG. 7).

1. A peptide having IPLRGAFINGRWDSQCHRFSNGAIACA (SEQ ID NO: 1) or variant thereof.

2. The peptide of claim 1 having at least one substitution, addition, or deletion such that the entire peptide is not naturally occurring.

3. The peptide of claim 1 having at least one molecular modification such that the peptide contains a non-naturally amino acid.

4. A non-naturally occurring derivative of a peptide having SEQ ID NO: 1 or variant thereof.

5. The non-naturally occurring derivative of claim 4 in the form of a prodrug.

6. The non-naturally occurring derivative of claim 4 wherein an amino, carboxyl, hydroxyl, or thiol group is substituted.

7. A recombinant vector comprising a nucleic acid encoding SEQ ID NO: 1 or variant thereof and a heterologous nucleic acid sequence.

8. An expression system comprising a recombinant vector of claim 7.

9. A cell comprising a recombinant vector of claim 7.

10. A pharmaceutical composition comprising a peptide having SEQ ID NO: 1, variant, or derivative thereof and a pharmaceutically acceptable excipient.

11. The pharmaceutical composition of claim 10 in the form of a capsule, tablets, pill, powder, or granule.

12. The pharmaceutical composition of claim 10 in the form of a sterilized pH buffered aqueous salt solution.

13. The pharmaceutical composition of claim 10 in the form of a container configured to spray a liquid or sealed container with a propellant.

14. A method of treating a viral infection comprising administering an effective amount of a pharmaceutical composition of claim 10 to a subject in need thereof.

15. The method of claim 11, wherein the virus is an influenza virus subtype H1.
